(19) United States
(12) Patent Application Publication
(10) Pub. No.: US 2014/0163064 A1
(43) Pub. Date: Jun. 12, 2014

(54) Method for Alleviating Symptoms of Urinary Incontinence

(71) Applicant: Eaton Scientific Systems, Ltd., Santa Monica, CA (US)

(72) Inventors: Hootan Melamed, Beverly Hills, CA (US); Edward W. Withrow, III, Santa Monica, CA (US); Jennifer R. Berman, Los Angeles, CA (US)

(73) Assignee: Eaton Scientific Systems, Ltd., Santa Monica, CA (US)

(21) Appl. No.: 14/181,452

(22) Filed: Feb. 14, 2014

Related U.S. Application Data
(63) Continuation-in-part of application No. 11/523,975, filed on Sep. 19, 2006, now Pat. No. 8,653,145.

(51) Int. Cl. A61K 31/46

(52) U.S. Cl. CPC A61K 31/46
USPC 514/304

(57) ABSTRACT

A method for alleviating urinary incontinence, by administering to a subject, including, but not limited to, climacteric women, a therapeutically effective amount of an anticholinergic agent. The anticholinergic agent may be an anti-muscarinic agent, and preferably, the anti-muscarinic agent may be homatropine, including its methylbromide salt.

### CROSS-REFERENCE TO RELATED APPLICATIONS

This patent application is a continuation-in-part application of U.S. patent application Ser. No. 11/523,975, filed on Sep. 19, 2006, which claims the benefit of U.S. Provisional Patent Application No. 60/719,756, filed on Sep. 22, 2005, which applications are incorporated herein by their entirety by this reference.

### TECHNICAL FIELD

This invention relates to a control of bladder function, for example, for those suffering from urinary incontinence or in climacteric women, among others, by administering an anticholinergic agent, and, in particular, by administering homatropine.

### BACKGROUND OF THE INVENTION

Urinary incontinence—the loss of bladder control—is a common and often embarrassing problem. The severity of urinary incontinence ranges from occasionally leaking urine when one coughs or sneezes; to having an urge to urinate that is often so sudden and strong that one cannot reach an appropriate restroom facility in time.

Urinary incontinence is the inability to control the release of urine from the bladder. Some people experience occasional, minor leaks or dribbles of urine. Others wet their clothes frequently.

The present application deals not so much with temporary incontinence often caused by consumption of alcohol or caffeine, or by a urinary tract infection, but with more persistent incontinence that tends to cause disruption of a person's daily life on a longer term basis.

According to the Mayo Clinic's internet website located at: http://www.mayoclinic.com/health/urinary-incontinence/DS00404/DSECTION=symptoms (assessed October 2012), there are several types of urinary incontinence, some of which are described below.

Stress incontinence is a loss of urine when pressure or stress is exerted on the bladder by coughing, sneezing, laughing, exercising, or heavy lifting. Stress incontinence occurs when the sphincter muscle of the bladder is weakened. In women, physical changes resulting from pregnancy, childbirth and menopause can cause stress incontinence. In men, removal of the prostate gland can lead to stress incontinence.

Urge incontinence is characterized by a sudden, intense urge to urinate, followed by an involuntary loss of urine. The bladder muscle contracts and may provide a brief warning to reach a toilet (seconds to a few minutes). With urge incontinence, a person may need to urinate often, including throughout the night. Urge incontinence may be caused by urinary tract infections, bladder irritants, bowel problems, Parkinson's disease, Alzheimer's disease, stroke, injury, or nervous system damage associated with multiple sclerosis. Urge incontinence is often called “overactive bladder.”

Overflow incontinence is characterized by frequent or constant dribble or urine. Overflow incontinence is typically caused by an inability to empty the bladder. At times, a person may feel as though he or she never completely empties his or her bladder. When urination is attempted, typically only a weak stream of urine is produced. This type of incontinence may occur in people with a damaged bladder, blocked urethra, or nerve damage from diabetes, multiple sclerosis or spinal cord injury. In men, overflow incontinence is often associated with prostate gland problems.

Overactive bladder is a urological condition characterized, at least in part, by symptoms that may overlap with symptoms with various forms of urinary incontinence, particularly urge incontinence. According to the Mayo Clinic, “[o]veractive bladder is a problem with bladder storage function that causes a sudden urge to urinate. The urge may be difficult to suppress, and overactive bladder can lead to the involuntary loss of urine (incontinence)” (http://www.mayoclinic.com/health/overactive-bladder/DS00827). The sudden urge to urinate is believed to be caused, in most cases, by spasms or involuntary contraction of bladder muscles.

Accordingly, while there may not be absolute overlap between the broad concept of urinary incontinence and the condition of overactive bladder, it is nonetheless clear that persons of ordinary skill in the art recognize that there are aspects of each that overlap such that it is generally agreed that overactive bladder is a form of urge incontinence.

Common causes of persistent urinary incontinence include, but are not limited to: a prolapsed pelvic floor in women who have had a vaginal child delivery; a weakened bladder muscles as a result of aging that reduce the ability of the bladder to store urine; a reduction in estrogen levels in peri-menopausal and menopausal women (because estrogen is believed to maintain bladder and urethral health in women); a hysterectomy, which can damage pelvic floor muscles and lead to incontinence; benign prostate hyperplasia; prostrate or bladder cancer; and, obstructions in the urinary system (such as stones in the bladder or ureter).

Treatment of urinary incontinence often depends on the type of incontinence, the severity of the condition, and the underlying cause of the condition. Treatments may include behavioral techniques such as bladder training, physical therapy such as pelvic floor muscle exercises, or utilization of medical devices such as a urethral insert or a pessary. In these or other situations, the person suffering from urinary incontinence may find it necessary to wear a pad or diaper to alleviate the outward manifestations of urinary incontinence.

Another mode of treatment of urinary incontinence and overactive bladder includes pharmaceutical intervention. Typical classes of drugs used to treat urinary incontinence and overactive bladder include anticholinergics, as well as, for certain forms of incontinence, estrogen, and certain antidepressants such as imipramine and duloxetine (Cymbalta®).

Anticholinergics commonly used to treat urge incontinence and overactive bladder include oxybutynin (Ditropan®), tolterodine (Detrol®), darifenacin (Enablex®), fesoterodine (Toviaz®), solifenacin (Vesicare®) and trospium (Sanctura®). Typical side effects of these medications include dry mouth, constipation, blurred vision, flushing, dizziness, and drowsiness.

In mammals, the cholinergic system includes two main classes of cell surface receptors—nicotinic and muscarinic. Within each main class of nicotinic and muscarinic receptors, there are some of many subtypes.

Nicotinic receptors, which are part of the neurotransmitter-gated ion channel superfamily, are found throughout the nervous system, but are classically found in skeletal muscle. Specifically, nicotinic receptors are acetylcholine-grated cation channels.

Muscarinic receptors, including their subtypes M1-M5, are members of the seven-transmembrane domain, G-protein-coupled receptor superfamily. While rigid classifications may not be possible, it is generally the case that the odd-numbered muscarinic receptors couple through G proteins, G_{q} or G₁₁, to phospholipase C, leading to production of diacylglycerol and inositol 1,4,5-trisphosphate, and their subsequent effects, including increases in intracellular Ca²⁺ concentration and activation of protein kinase C.

The even-numbered muscarinic receptors are typically coupled through G proteins, Gᵢ or Gₒ, to adenylyl cyclase, leading to inhibition of adenylyl cyclase activity and a reduction in intracellular cAMP levels.

Anticholinergics used for treatment of urinary incontinence and overactive bladder are anti-muscarinic agents. It is hypothesized that control of bladder function in humans is largely mediated by M2 and M3 receptors, although the precise mechanism is not completely understood. Thus, anti-muscarinics are frequently used for bladder control in subjects experiencing incontinence and/or overactive bladder, the latter being a form of urge incontinence.

While it is clear from the foregoing that both men and women can suffer from urinary incontinence/overactive bladder for a variety of reasons, it is similarly clear that peri-menopausal and menopausal women (i.e., climacteric women) may experience urinary incontinence and/or overactive bladder based on a reduction in estrogen levels inasmuch as estrogen is believed to maintain bladder and urethral health in women, and the reduction of estrogen levels in peri-menopausal and menopausal women may have deleterious effects on bladder and urethral health such that they experience varying levels of urinary incontinence and/or overactive bladder.

Climacteric is defined as the syndrome of endocrine, somatic, and psychological changes occurring at the termination of the reproductive period in the female. According to the Greene Climacteric scale (Greene (1998) Maturitas 29:25-31), there are 21 common symptoms associated with a woman's climacteric stage, namely heart beating quickly or strongly, feeling tense or nervous, difficulty in sleeping, excitability, attacks of panic, difficulty in concentrating, feeling tired or lacking in energy, loss of interest in most things, feeling unhappy or depressed, crying spells, irritability, feeling dizzy or faint, pressure or tightness in head or body, parts of the body feel numb or tingling, headaches, muscle and joint pains, loss of feeling in hands and feet, breathing difficulties, hot flushes, sweating at night, and loss of interest in sex. Other symptoms commonly experience in climacteric women include urinary frequency, urgency, and incontinence, as well as palpitations, and anxiety.

Generally speaking, overstimulation of the muscarinic receptors leads to diarrhea, frequent urination, mitosis, bradycardia, bronchorrhea, emesis, lacrimation, and salivation. Other symptoms resulting from overstimulation of the receptors include nausea, vomiting, as well as eye pain, and blurred or dim vision. Similarly, nicotinic stimulation causes muscle pain, tremors, weakness, hypertension, and fasciculations. Advantageously, anticholinergic agents result in antimuscarinic and antinicotinic actions. For example, anticholinergic agents are routinely given to people with urinary incontinence to prevent frequent urination (see, e.g., U.S. Pat. No. 6,919,092).

It is believed that anticholinergics act peripherally and not in the central nervous system, i.e., hypothalamus, to block the muscarinic receptors located on tissues which receive parasympathetic postganglionic nerves. One exception is the sweat glands; which receive sympathetic-cholinergic nerves. The hypothalamus is one of several brain areas that regulate the discharge rate of parasympathetic and sympathetic nerves by descending nerve fibers that synapse with either the parasympathetic preganglionic or sympathetic preganglionic nerves. It appears that the hypothalamus is the major brain area that regulates the discharge rate of the autonomic nerves (which may increase or decrease). The pathophysiology of how the decrease in estrogen affects the hypothalamic regulation of body temperature is largely unknown. However, not wishing to be bound by theory, it is believed that the anticholinergic agent disclosed herein modulates hypothalamic regulation of body temperature via muscarinic receptor activity thereby reducing climacteric symptoms such as hot flushes and urinary incontinence in climacteric subjects.

At present, while antimuscarinics are used to treat urinary incontinence/overactive bladder, these drugs are not free of the aforementioned side effects such as dry mouth, constipation blurred vision, and flushing, as well as possible drowsiness. In addition, many of the current drugs have significant drug interactions, which make them less desirable for older persons who are often on a number of medications for different conditions.

Accordingly, there is a need in the art for effective treatment regimes for relieving urinary incontinence in its various forms, including symptoms of the climacteric. The present invention meets this long-felt need.

### SUMMARY OF THE INVENTION

The present invention is a method for alleviating urinary incontinence, including in climacteric subjects suffering from climacteric symptoms. The method involves administering to a subject in need thereof a therapeutically effective amount of an anticholinergic agent thereby alleviating the urinary incontinence in the subject. In some embodiments, the anticholinergic agent is homatropine, or a salt thereof.

In another aspect of the invention, the present invention comprises a method for alleviating at least one climacteric symptom or condition, namely urinary incontinence by administering to a climacteric subject a therapeutically effective amount of an anticholinergic agent, thereby alleviating the incontinence in the climacteric subject.

In another aspect, the present invention comprises a method for alleviating urinary incontinence by administering to a subject a therapeutically effective amount of an anti-muscarinic agent, thereby alleviating the incontinence in the subject.

More specifically, the present invention comprises a method for alleviating urinary incontinence by administering to a subject a therapeutically effective amount of homatropine, thereby alleviating the incontinence in the subject.

Even more specifically, the present invention comprises a method for alleviating urinary incontinence by administering to a subject a therapeutically effective amount of homatropine methylbromide, thereby alleviating the incontinence in the subject.

### DETAILED DESCRIPTION OF PRESENTLY PREFERRED EMBODIMENTS OF THE INVENTION

The detailed description set forth below is intended as a description of presently-preferred embodiments of the invention and is not intended to represent the only forms in which the present invention may be composed or utilized. The description sets forth the functions and the sequence of steps for composing and operating the invention in connection with the illustrated embodiments. It is to be understood, however, that the same or equivalent functions and sequences may be accomplished by different embodiments that are also intended to be encompassed within the spirit and scope of the invention.

The present invention is a method for alleviating urinary incontinence in a subject by administering to the subject a therapeutically effective amount of an anticholinergic agent, thereby alleviating the urinary incontinence. More specifically, the present invention comprises a method for alleviating at least one climacteric symptom, namely urinary incontinence, by administering to a climacteric subject a therapeutically effective amount of an anticholinergic agent, thereby alleviating the urinary incontinence symptoms in the climacteric subject. The present invention also comprises a method for alleviating urinary incontinence by administering to a subject a therapeutically effective amount of an anticholinergic agent, thereby alleviating the urinary incontinence symptoms in the climacteric subject. The present invention may also be extended beyond climacteric subjects to anyone who is experiencing symptoms of urinary incontinence.

As used in the context of the present invention, “an anticholinergic agent” can be a compound that acts as an antagonist at the muscarinic receptor. In particular, the muscarinic receptor can be M1 and/or M2 muscarinic receptors, as well as M3 muscarinic receptors, or other muscarinic receptors. In particular embodiments, the anticholinergic agent can be a belladonna alkaloid including, but not limited to, atropine, scopolamine, methscopolamine, homatropine, hyoscyamine, wherein these compounds are normally administered as a salt, i.e., tertiary amines. For example, the atropine can be selected from a group consisting of atropine sulfate, atropine oxide, atropine-HCl salt, and methylatropine nitrate. The scopolamine can be selected from a group consisting of hydrobromide salt and methylbromide salt of scopolamine. The homatropine can be selected from a group consisting of hydrobromide salt and methylbromide salt of homatropine. The hyoscyamine can be selected from a group consisting of hydrobromide salt and sulfate salt of hyoscyamine. These agents, particularly the salt forms thereof, are readily available from a number of commercial sources or can be made or prepared according to standard methods well-known in the art. Salt forms of the identified anticholinergic agents are identified as follows:

Atropine, CAS-51-55-8 or CAS-51-48-1 (anhydrous form); atropine sulfate, CAS-59-8-99-6; atropine oxide, CAS-4438-22-6 or its HCl salt, CAS-4574-60-1; and methylatropine nitrate, CAS-52-88-0.

Homatropine, CAS-87-00-3; hydrobromide salt, CAS-51-56-9; methylbromide salt, CAS-80-49-9.

Hyoscyamine (d, l), CAS-101-31-5; hydrobromide salt, CAS-306-03-6; and sulfate salt, CAS-6835-16-1.

Scopolamine, CAS-51-34-3; hydrobromide salt, CAS-6533-68-2; methylbromide salt, CAS-155-41-9.

Other anticholinergic agents include ipratropium (e.g., as the bromide), sold under the name ATROVENT; oxitropium (e.g., as the bromide); and tiotropium (e.g., as the bromide) (CAS-139404-48-1). Also of interest are methantheline (CAS-53-46-3), propantheline bromide (CAS-50-34-9), anisotropine methyl bromide or Valpin 50 (CAS-80-50-2), clidinium bromide (QUARZAN, CAS-3485-62-9), isopropamide iodide (CAS-71-81-8), mepenzolate bromide (U.S. Pat. No. 2,918,408), tridihexethyl chloride (CAS-4310-35-4), and hexocyclium methylsulfate (CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO 01/04118 for other exemplary anticholinergic agents.

In particular embodiments, the anticholinergic agent is homatropine, or a salt thereof. While the hydrobromide salt of homatropine is well-known for use in ophthalmology as a cycloplegic and mydriatic, and the 8-methyl derivative of homatropine hydrobromide is a well-known oral therapeutic for use as an antispasmodic and inhibitor of secretions, especially in gastrointestinal disorders, homatropine (including the hydrobromide and methylbromide salts) has not been described in the art for use in alleviating climacteric symptoms, including urinary frequency, urgency, and incontinence.

It is believed that homatropine methylbromide does not pass the blood-brain barrier, which may serve to minimize potential side effects from its use relative to other anti-muscarinics used to treat urinary incontinence that do cross the blood-brain barrier and may cause dizziness and/or drowsiness, as well as other potential side-effects that are CNS-based.

The amount of anticholinergic agent or salt thereof which is required to achieve a therapeutic effect will, of course, vary with the particular agent, the route of administration, and the subject under treatment. The compounds of the invention can be administered in a dose ranging from approximately 0.005 mg to approximately 100 mg per day, or more suitably approximately 0.05 g to approximately 100 mg per day, approximately 0.05 mg to approximately 50 mg per day.

In accordance with the instant method, a therapeutically effective amount of an anticholinergic agent, such as a belladonna alkaloid, and in particular, homatropine or salt thereof, can be administered to a climacteric subject, which includes peri-menopausal and post-menopausal women, wherein said effective amount alleviates, reduces, or ameliorates at least one climacteric symptom in the subject. Accordingly, homatropine, or salt thereof, can be administered in a dose ranging from approximately 0.005 mg to approximately 100 mg per day, or more suitably approximately 0.05 mg to approximately 100 mg per day, or 0.05 mg to 50 mg per day.

Climacteric symptoms which can be alleviated by the anticholinergic agent include rapid heartbeat, strong heartbeat, feeling tense, feeling nervous, difficulty in sleeping, excitability, attacks of panic, difficulty in concentrating, feeling tired, lacking in energy, loss of interest in most things, feeling unhappy, feeling depressed, crying spells, irritability, feeling dizzy, feeling faint, pressure in head, pressure in body, tightness in head, tightness in body, numbness in a body part, tingling in a body part, headaches, muscle pains, joint pains, loss of feeling in hands, loss of feeling in feet, breathing difficulties, hot flushes, sweating at night, loss of interest in sex, and urinary frequency, urgency, and/or incontinence. In some embodiments, the anticholinergic agent alleviates climacteric symptoms resulting from overstimulation of the muscarinic receptors. In particular embodiments, the muscarinic receptor is the M1, M2, or M3 receptor. In other embodiments, the anticholinergic agent alleviates urinary frequency, urgency, and/or incontinence. In a preferred embodiment, homatropine or salt thereof can be administered to alleviate urinary frequency, urgency, and/or incontinence. In still other embodiments, the anticholinergic agent generally improves the quality of life.

While it is possible for the anticholinergic agent or salt thereof to be administered alone, it is generally desirable to present it as a pharmaceutical formulation. Accordingly, the present invention further provides a method for alleviating at least one climacteric symptom, in particular, urinary frequency, urgency, and/or incontinence, by administering to the climacteric subject a therapeutically effective amount of an anticholinergic agent, wherein the anticholinergic agent, such as belladonna alkaloid, in particular, homatropine, is administered in admixture with a pharmaceutically acceptable carrier, and optionally one or more other therapeutic ingredients. Another embodiment provides a method for alleviating at least one climacteric symptom, in particular, urinary frequency, urgency, and/or incontinence, by administering to the climacteric subject a therapeutically effective amount of an anticholinergic agent, wherein the anticholinergic agent, such as belladonna alkaloid, in particular, homatropine, is administered in admixture with an excipient. Another embodiment provides for a method for alleviating at least on climacteric symptom, in particular, urinary frequency, urgency, and/or incontinence, by administering to the climacteric subject a therapeutically effective amount of anticholinergic agent, such as belladonna alkaloid, in particular, homatropine, via oral, parenteral (including subcutaneous, intradermal, intramuscular, intravenous and intraarticular), intranasal, inhalation (including fine particle dusts or mists which may be generated by means of various types of metered dose pressurized aerosols, nebulisers or insufflators), rectal and topical (including dermal, buccal, sublingual and intraocular) administration although the most suitable route may depend upon for example the condition and symptom of the recipient subject. The formulations can conveniently be presented in unit dosage form and can be prepared by any of the methods well-known in the art of pharmacy. See, for example, Remington: The Science and Practice of Pharmacy, Alfonso R. Gennaro, editor, 20th ed. Lippincott Williams & Wilkins: Philadelphia, Pa., 2000. Accordingly, an embodiment of this invention is a method for alleviating at least one climacteric symptom in a climacteric subject by administering to the climacteric subject a therapeutically effective amount of an anticholinergic agent, thereby alleviating at least one climacteric symptom in the climacteric subject, wherein the anticholinergic agent, such as belladonna alkaloid, and in particular, homatropine, is administered via a mode selected from a group consisting of oral, parenteral, intranasal, inhalation, rectal and topical administration.

Suitable methods for preparing formulations include the step of bringing the active ingredient (i.e., the anticholinergic agent) into association with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, lubricating, surface active or dispersing agent. Molded tablets can be made by molding in a suitable machine a mixture of the powdered compound moistened with an inert liquid diluent. The tablets can optionally be coated or scored and can be formulated so as to provide slow or controlled release of the active ingredient therein.

Formulations for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. The formulations can be presented in unit-dose or multi-dose containers, for example sealed ampoules and vials, and can be stored in a freeze-dried (lyophilized) condition requiring only the addition of the sterile liquid carrier, for example saline or water-for-injection, immediately prior to use.

Extemporaneous injection solutions and suspensions can be prepared from sterile powders, granules and tablets of the kind previously described.

Dry powder compositions for topical delivery to the lung by inhalation can, for example, be presented in capsules and cartridges of, for example, gelatin, or blisters, of for example, laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge can contain the active ingredient in combination with another therapeutically active or inactive ingredient. Alternatively, the compound of the invention can be presented without excipients. Packaging of the formulation can be suitable for unit dose or multi-dose delivery. In the case of multi-dose delivery, the formulation can be pre-metered (e.g., as in DISKUS, see GB 2242134 or DISKHALER, see GB 2178965, GB 2129691 and GB 2169265) or metered in use (e.g., as in TURBUHALER, see EP 69715). An example of a unit-dose device is ROTAHALER (see GB 2064336). The DISKUS inhalation device comprises an elongate strip formed from a base sheet having a plurality of recesses spaced along its length and a lid sheet hermetically but peelably sealed thereto to define a plurality of containers, each container having therein an inhalable formulation containing an anticholinergic agent of the invention preferably combined with lactose. Desirably, the strip is sufficiently flexible to be wound into a roll. The lid sheet and base sheet will preferably have leading end portions which are not sealed to one another and at least one of the said leading end portions is constructed to be attached to a winding means. Also, the hermetic seal between the base and lid sheets extends over their whole width. The lid sheet can desirably be peeled from the base sheet in a longitudinal direction from a first end of the said base sheet.

Spray compositions for topical delivery to the lung by inhalation can, for example, be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain the anticholinergic agent optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluloromethane, trichlorofluoromethane, dichlorotetrafluoroethane, especially 1,1,1,2-tetrafluoroethane, 1,1,1,2,3,3,3-heptafluoro-n-propane or a mixture thereof. Carbon dioxide or other suitable gas can also be used as propellant. The aerosol composition can be excipient free or can optionally contain additional formulation excipients well-known in the art such as surfactants, e.g., oleic acid or lecithin and cosolvents, e.g., ethanol. Pressurized formulations will generally be retained in a canister (e.g., an aluminum canister) closed with a valve (e.g., a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced can be size reduced by conventional means e.g., by micronisation. The desired fraction can be separated out by air classification or sieving. Preferably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

Solutions for inhalation by nebulation can be formulated with an aqueous vehicle with the addition of agents such as acid or alkali, buffer salts, isotonicity adjusting agents or antimicrobials. They can be sterilized by filtration or heating in an autoclave, or presented as a non-sterile product.

Formulations for rectal administration can be presented as a suppository with the usual carriers such as cocoa butter or polyethylene glycol.

Formulations for oral administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose an acacia.

Preferred unit dosage formulations are those containing an effective dose, as hereinbefore recited, or an appropriate fraction thereof, of the active ingredient.

One embodiment of the present invention comprises treating at least one climacteric symptom or condition, namely urinary incontinence, by administering to a climacteric subject a therapeutically effective amount of an anticholinergic agent, thereby alleviating the incontinence in the climacteric subject.

Another embodiment of the present invention comprises treating a subject experiencing urinary incontinence by administering to the subject a therapeutically effective amount of an anti-muscarinic agent, thereby alleviating the incontinence in the subject.

Still another embodiment of the present invention comprises treating a subject experiencing urinary incontinence by administering to the subject a therapeutically effective amount of homatropine, thereby alleviating the incontinence in the subject.

Still yet another embodiment of the present invention comprises treating a subject experiencing urinary incontinence by administering to the subject a therapeutically effective amount of homatropine methylbromide, thereby alleviating the incontinence in the subject.

For purposes of the present invention, it is to be understood that urinary incontinence can be broken down into a number of subtypes (e.g., stress incontinence, overflow incontinence, and urge incontinence), and also that overactive bladder is often categorized as a form of urge incontinence in which muscle spasms in and/or around the bladder lead to increased urgency and frequency of urination.

The foregoing description of the preferred embodiments of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

What is claimed is:

1. A method for treating urinary incontinence, comprising:
administering to a subject a therapeutically effective amount of homatropine, thereby alleviating urinary incontinence in the subject.

2. The method of claim 1, wherein the homatropine is administered in admixture with a pharmaceutically acceptable carrier.

3. The method of claim 1, wherein the homatropine is administered in admixture with a excipient.

4. The method of claim 1, wherein the homatropine is administered via a mode selected from a group consisting of oral, parenteral, intranasal, inhalation, rectal and topical administration.

5. The method of claim 1, wherein the urinary incontinence is selected from the group consisting of stress incontinence, overflow incontinence, urge incontinence, and overactive bladder.

6. A method for treating urinary incontinence, comprising:
administering to a subject a therapeutically effective amount of homatropine methylbromide, thereby alleviating urinary incontinence in the subject.

7. The method of claim 6, wherein the homatropine methylbromide is administered in admixture with a pharmaceutically acceptable carrier.

8. The method of claim 6, wherein the homatropine methylbromide is administered in admixture with an excipient.

9. The method of claim 6, wherein the homatropine methylbromide is administered via a mode selected from a group consisting of oral, parenteral, intranasal, inhalation, rectal and topical administration.

10. The method of claim 6, wherein the urinary incontinence is selected from the group consisting of stress incontinence, overflow incontinence, urge incontinence, and overactive bladder.

11. A method for treating urinary incontinence in a climacteric subject, comprising:
administering to a subject a therapeutically effective amount of homatropine, thereby alleviating urinary incontinence in the subject.

12. The method of claim 11, wherein the homatropine is administered in admixture with a pharmaceutically acceptable carrier.

13. The method of claim 11, wherein the homatropine is administered in admixture with an excipient.

14. The method of claim 11, wherein the homatropine is administered via a mode selected from a group consisting of oral, parenteral, intranasal, inhalation, rectal and topical administration.

15. The method of claim 11, wherein the urinary incontinence is selected from the group consisting of stress incontinence, overflow incontinence, urge incontinence, and overactive bladder.

16. A method for treating urinary incontinence in a climacteric subject, comprising:
administering to a subject a therapeutically effective amount of homatropine methylbromide, thereby alleviating urinary incontinence in the subject.

17. The method of claim 16, wherein the homatropine methylbromide is administered in admixture with a pharmaceutically acceptable carrier.

18. The method of claim 16, wherein the homatropine methylbromide is administered in admixture with an excipient.

19. The method of claim 16, wherein the homatropine methylbromide is administered via a mode selected from a group consisting of oral, parenteral, intranasal, inhalation, rectal and topical administration.

20. The method of claim 16, wherein the urinary incontinence is selected from the group consisting of stress incontinence, overflow incontinence, urge incontinence, and overactive bladder.
